(19) United States
(12) Patent Application Publication
(10) Pub. No.: US 2011/0136244 A1
(43) Pub. Date: Jun. 9, 2011

(54) METHODS AND KITS FOR A COLOR CHANGING SAMPLE DILUENT

(76) Inventor: Mark James Kopnitsky, Lenhartsville, PA (US)

(21) Appl. No.: 12/811,906

(22) PCT Filed: Jan. 28, 2009
(86) PCT No.: PCT/US09/32176

§ 371 Date: Feb. 20, 2011

Related U.S. Application Data
(60) Provisional application No. 61/023,935, filed on Jan. 28, 2008.

(51) Int. Cl. G01N 33/50; G01N 21/00

(52) U.S. Cl. 436/86; 436/164

(57) ABSTRACT

The present invention provides methods and kits in assessing color change in a series of reaction mixes while providing significant improvement in the amount of non-specific binding. The method provides for a dramatic color change when incorporated into a series of assays or used in an individual reaction. The color change enables quick visual tracking and verification of the addition of reaction components. Further, the color change allows for verification that an instrument is performing correctly in reagent addition during an automated assay. In one embodiment, the reaction mix is initially a yellowish color. Upon adding serum or plasma, the resulting solution undergoes a dramatic color change to blue-purple, allowing for easy visual verification. Color change depends upon pH shifts in the sample preparation upon addition of serum or plasma. The method is useful whenever two or more reagents are combined and confirmation of the combination is required.

### CROSS REFERENCE TO RELATED APPLICATIONS

This application is a non-provisional application, which incorporates by reference herein and claims priority, in part, of U.S. Provisional Application No. 61/023,935, filed 28 Jan. 2008.

### BACKGROUND

1. Field of the Invention

The present invention relates to a testing methodology utilizing a chemical reaction for the colorimetric assessment of a reaction mixture. More specifically, methods and kits suitable for use in testing systems are presented for color detection based upon a change in pH for a specimen diluent in serum samples.

1. Background Art

There are known hemoglobin detecting test systems that employ color detection, for example, U.S. Pat. No. 4,386,053 to Motobayashi focuses on the use of boric esters to retard the premature reaction of hydroperoxide and the indicator in a hemoglobin-detecting test system. U.S. Pat. No. 5,089,420 to Albarella similarly describes a method for detecting hemoglobin which uses amine borate complexes for the purpose of blocking the same mutual incompatibility of the two reagents, as Motobayashi sought to effect. In neither case is a pH indicator employed or is a color change strictly dependent upon a pH shift in the sample with the addition of serum or plasma. U.S. Pat. No. 5,397,710 to Steinman is directed toward a dry indicator composition capable of exhibiting a sufficient color transition upon contracting a biological fluid test sample to demonstrate the present or concentration of magnesium ions in the test sample.

Various processes for measuring such endpoints as ionic strength or specific gravity of liquid samples by color change or absorbance have been disclosed. For example, U.S. Pat. No 4,015,462 discloses a process comprising holding microcapsules encapsulating colorants on a carrier matrix, contacting the microcapsules with a solution of low osmotic pressure to raise the inner pressure of microcapsules to distend and rupture the microcapsules and release the colorants and applying a phenomenon of changing the color of matrix. U.S. Pat, No. 4,473,6f50 discloses a reagent composition comprising a weakly basic polyelectrolyte polymer neutralized with a strong organic acid, and an indicator, said polyelectrolyte polymer being polyethyleneimine, polyvinyiarnine, polyaminostyrene, or a copolymer of the monomers constitution the polyelectrolyte polymer. U.S. Pat. No. 5,106,752 and U.S. Pat. No, 5,064,615 disclose a composition comprising at least one pH buffer without a polyelectrolyte polymer or at least one pH buffer and/or at least one complex-forming agent, and at least one pH indicator.

Accordingly, It is desirable to devise a test that would allow a visual verification of the addition of a reagent to a reaction mixture. Further, the indicators in the reaction mixture should he designed to minimize the amount of non-specific binding. In contrast to the above-described methodologies of the art, the instant invention is advantageous for many reasons including that it does not employ hydroperoxide, does not test a redox reaction, and does include visual detection of a pH indicator.

### SUMMARY OF THE INVENTION

The present invention provides methods and kits for a system used in assessing color change in a series of reaction mixes while providing significant improvement in the amount of non-specific binding. The system comprises a visual pH indicator and a reaction mixture wherein a dramatic color change appears when a selected reagent is combined or added to the mixture. The color change enables quick visual tracking and verification of the addition of reaction components. Further, the color change allows for verification that an instrument is performing correctly with respect to reagent addition during an automated assay. in one embodiment, the reaction mix is initially a yellowish color. Upon adding serum or plasma, the resulting solution undergoes a dramatic color change to blue-purple, allowing for easy visual verification. Color change depends upon pH shifts in the sample preparation upon addition of serum or plasma. The method is useful whenever two or more reagents are combined and confirmation of the combination is required. To ensure that the combination is accomplished, the resulting solution is formulated to exhibit a totally unique visual color. With appropriate spectrophotometric specifications established to verify, the method can be automated for instrument use.

### DETAILED DESCRIPTION OF THE INVENTION

In general, serum or plasma exhibit ability to function as strong buffers. The present invention provides for a specimen diluent that is a very weak buffer. The pH of this buffer is made to be considerably lower than that of normal human serum (pH, approximately 5.5). A pH indicator such as, but not limited to, bromocresol purple is added, This indicator transitions color around pH 6. So while in an acidic state it is yellow, but upon transition to neutral or basic it turns purple/blue.

This characteristic provides an advantage for manual or automated addition of selected reagents containing serum or plasma to the diluent. Provided the pH shifts from approximately 5.5 to approximately 7.0, the resulting reaction solution will change color, signifying addition and confirmation of the presence of the reagent.

The present invention includes similar formulations using other pH indicators or protein detection chemicals. For each of these embodiments, a solution is created that differs in some way from normal serum or plasma and then when the serum is added the color characteristics of the solution change in a manner that some chemical in the original solution undergoes a visual change.

The method can be applied in other applications where clinical specimens are “diluted” prior to processing. For example, clinical specimens such as, but not limited to, urine, sputum, fecal samples or cerebral spinal fluid are considered to be included in the scope of the present invention. In the broadest perspective of the present invention, any two or more reagents may be combined as part of an assay. A test is performed by combining at lease reagent A and reagent B. To ensure that the reagents are combined, the resulting solution exhibits a totally unique visual color, providing confirmation of the combination.

Examples of potential formulations in accordance with the present invention include, but are not limited to, the reagents/components shown in Table 1 and Table 2.

TABLE 1

| Component | Quantity Needed | Units | Manufacturer |
|---|---|---|---|
| Na2HPO4 | 0.0296 | grams | J T Baker |
| KH2PO4 | 0.0086 | grams | Sigma |
| NaCl | 6.6 | grams | J T Baker |
| Poly (vinyl alcohol) | 1.000 | grams | Sigma |
| Bromocresol Purple | 0.100 | grams | Sigma |
| Bovine Serum Albumin | 0.010 | grams | Sigma |
| Proclin 950 | 0.500 | mLs | Supelco |
| Normal Goat Serum | 6.250 | mLs | Fitzgerald |
| Tween 20 | 20.000 | mLs | Sigma |
| Casitone 30% sol. | 6.600 | mLs | N/A |
| NaN3 (Azide) | 1.00 | grams | Sigma |
| Distilled water* | To one Liter | mLs | VWR |
| 5M HCl | TBD | mLs | J. T Baker |

*Formulation is per Liter, final volume

TABLE 2

| Component | Quantity Needed | Units | Manufacturer |
|---|---|---|---|
| Na2HPO4 | 0.0296 | grams | J T Baker |
| KH2PO4 | 0.0086 | grams | Sigma |
| NaCl | 6.6 | grams | J T Baker |
| Bromocresol Purple | 0.100 | grams | Sigma |
| Bovine Serum Albumin | 0.010 | grams | Sigma |
| Proclin 950 | 0.500 | mLs | Supelco |
| Tween 20 | 20.000 | mLs | Sigma |
| Casitone 30% sol. | 6.600 | mLs | N/A |
| NaN3 (Azide) | 1.00 | grams | Sigma |
| Distilled water* | To one liter | mLs | VWR |
| 5M HCl | TBD | mLs | J. T Baker |

*Formulation is per Liter, final volume

The scope of the present invention further includes any automated application know in the art, involving a distinctive color change during reagent addition. Further although the foregoing invention has been described in some detail for purposes of clarity of understanding, it will be readily apparent to those of ordinary skill in the art in light of the teachings of this invention that certain changes and modifications may be made thereto without departing from the spirit or scope of the invention.

What is claimed is:

1. A method for visually assessing a reaction in a fluid specimen comprising:
a. obtaining a fluid specimen;
b. combining a visual indicator with a diluent in a reaction mixture having the same physical property as the fluid specimen wherein visualization of said physical property is noticeably different without addition of said fluid specimen;
c. adding said fluid specimen to said diluent; and
d. visualizing an alteration in said physical property becomes wherein said alteration indicates a complete reaction.

2. The method of claim 1 wherein said fluid specimen is selected from a group consisting of urine, sputum, fecal sample, cerebral spinal fluid, blood, serum, plasma, and combinations thereof.

3. The method of claim 1 where said visual indicator is color.

4. The method of claim 3 wherein said color results from a change in pH.

5. The method of claim 4 wherein said change in pH results from the addition of bromocresol purple.

6. The method of claim 1 wherein said visual indicator is any protein detection chemical.

7. A colorimetric method for determining a chemical reaction for a serum sample in an automated instrument comprising:
a. obtaining a serum sample;
b. combining bromocresol purple with a diluent in a reaction mixture wherein the combination results in a yellow color;
c. adding said serum sample to said diluent wherein said addition results in a pH shift; and
d. visualizing a blue to purple change in said color, indicating a complete reaction.

8. A kit for visually assessing a reaction in a fluid specimen comprising:
a. a fluid specimen;
b. a visual indicator; and
c. a diluent having the same physical property as the fluid specimen wherein visualization of said physical property is noticeably different without addition of said fluid specimen.

9. The kit of claim 8 wherein said fluid specimen is selected from a group consisting of urine sputum fecal sample, cerebral spinal fluid, blood, serum, plasma, and combinations thereof.

10. The kit of claim 8 where said visual indicator is bromocresol

11. A kit for determining a chemical reaction for a fluid specimen in an automated instrument comprising:
a. a fluid specimen;
b. a diluent where said diluent contains a visual indicator; and
c. means for combining elements a and b, wherein the combination results in a change in color.

12. The kit in claim 11 wherein said fluid specimen is selected from a group consisting of urine sputum fecal sample, cerebral spinal fluid, blood, serum, plasma, and combinations thereof,

13. The kit of claim 11 where said visual indicator is bromocresol purple.
